(19) United States
(12) Patent Application Publication
(10) Pub. No.: US 2024/0115761 A1
(43) Pub. Date: Apr. 11, 2024

(54) IMPLANTS WITH SWELLABLE NANOCELLULOSE

(71) Applicant: BIOTRONIK AG, Buelach (CH)

(72) Inventors: Alexander Rzany, Nuernberg (DE); Verena Niopek, Erlangen (DE); Bernhard Hensel, Erlangen (DE)

(21) Appl. No.: 18/546,359

(22) PCT Filed: Feb. 16, 2022
(86) PCT No.: PCT/EP2022/053789

§ 371 Date: Aug. 14, 2023

(30) Foreign Application Priority Data
Feb. 24, 2021 (EP) 21158977.5

(51) Int. Cl. A61L 27/20

(52) U.S. Cl. CPC A61L 27/20; A61L 2430/20

(57) ABSTRACT

A method for producing a swellable body including bacterial cellulose includes providing a body made of bacterial cellulose, cleaning the body using at least one liquid medium, freezing the body at atmospheric pressure for at least six hours, freeze drying the body, and mechanically pressing the entire molded body or parts thereof after freeze drying of the body. A body of bacterial cellulose is improved regarding its swelling capability. A swelling factor of at least 1000% is provided by the body of bacetrial cellulose

### PRIORITY CLAIM

This application is a 35 U.S.C. 371 US National Phase and claims priority under 35 U.S.C. § 119, 35 U.S.C. 365(b) and all applicable statutes and treaties from prior PCT Application PCT/EP2022/053789, which was filed Feb. 16, 2022, which application claimed priority from European Patent Application Number 21158977.5, filed Feb. 24, 2021.

### FIELD OF THE INVENTION

A field of the invention is swellable bacterial cellulose bodies, methods for making swellable bacterial cellulose bodies and medical implants including swellable bacterial cellulose bodies.

### BACKGROUND

For a variety of minimally invasive implant applications, an exceptionally swellable biomaterial is advantageous, or such applications are made possible in the first place by such a material. Examples include sealing rings on implants such as TAVI prostheses to prevent paravalvular leaks, stent systems to stabilize aneurysms, closure systems for the atrial auricle in the left atrium, and closure systems of holes or puncture sites.

Swellable biological materials based on tissues of animal origin are known in the state of the art. Particularly, WO 2016/083351 A1 describes a swellable body made from bacterial cellulose.

### SUMMARY OF THE INVENTION

The invention provides a body of bacterial cellulose that is improved regarding its swelling capability. A swelling factor of at least 1000% is provided by the present body of bacetrial cellulose.

A method for producing a swellable body including bacterial cellulose includes providing a body made of bacterial cellulose, cleaning the body using at least one liquid medium, freezing the body at atmospheric pressure for at least six hours, freeze drying the body, and mechanically pressing the entire molded body or parts thereof after freeze drying of the body.

### BRIEF DESCRIPTION OF THE DRAWINGS

In the following, further features, advantages and embodiments of the present invention are explained with reference to the Figures, wherein

FIG. 1 shows a flow diagram of an embodiment of the method according to the present invention,

FIG. 2 shows photos of square sample patches (20 mm×20 mm) of a swellable body made from bacterial cellulose visualizing the described behavior of the material according to the invention,

FIG. 3 shows photos of ring samples visualizing the described behavior of the material according to the invention,

FIG. 4 shows such a medical implant in form of a TAVI prosthesis including a scaffold and a swellable body in form of an outer skirt (dashed) of the prosthesis, the body being produced with the method according to the present invention, and

FIG. 5 shows a cross-section of a plastically deformable cylindrical support structure (e.g. scaffold or stent) with a swellable body including one layer (top left) or two layers (top right) of swellable bacterial cellulose locally attached in a sector of the support structure. By swelling (bottom row), a cavity can be filled in this sector due to the extreme increase in thickness. The volume requirement can be expanded as desired by adding two or more layers of the material (bottom right).

### DETAILED DESCRIPTION OF THE PREFERRED EMBODIMENTS

Freeze drying is also known as lyophilization or cryodesiccation and corresponds to a low temperature dehydration process of freezing a body and lowering pressure of an atmosphere surrounding the body and removing the ice by sublimation to dry the body. The body formed with the method according to the present invention is herein also denoted as a material.

A swellable body produced using the method according to the present invention can be used in implants that incorporate an integrated seal for leakage, or are capable of filling cavities or seal openings. For this purpose, particularly, a special processing of bacterial cellulose, particularly nanocellulose, is used, which surprisingly leads to an enormous and unique swelling capacity.

Advantageously, the method for producing the swellable body for the production of a pressed, dry bacterial nanocellulose prevents the formation of intermolecular hydrogen bonds between the cellulose fibrils during drying or during pressing, the so-called cornification. Particularly, this results in an extremely high swelling capacity of more than 2000% by volume when coming in contact with an aqueous medium i.e. it becomes more than twenty times thicker, which is unique for pure bacterial cellulose. Advantageously, the method allows the production of virtually any three-dimensional molded part for use in a wide variety of implant classes. Furthermore, bacterial cellulose is a very blood- and biocompatible, non-degradable biomaterial, which is already approved in medical applications.

According to a further embodiment of the method, the entire body or parts thereof include a temperature of at least 20° C., and preferably up to 80° C., more preferably up to 50° C. upon mechanically pressing the entire body or parts thereof.

According to a further embodiment, the entire body or parts thereof are mechanically pressed with a pressure of at least 1 N/mm² and preferably up to 20 N/mm² and more preferably up to 10 N/mm².

Furthermore, according to an embodiment, the entire body or parts thereof are mechanically pressed for at least 15 minutes.

According to yet another embodiment of the method according to the present invention, the step of freezing the body includes subjecting the body for at least six hours to a temperature equal to or below −20° C. at atmospheric pressure (i.e. 1013.25 mbar±50 mbar).

Further, according to an embodiment, the step of freezing the body includes subjecting the body for at least six hours to a temperature equal to −45° C. or below −45° C. at atmospheric pressure (i.e. 1013.25 mbar±50 mbar). Further, in an embodiment, the step of freeze drying the body includes subjecting the body to a pressure of 0.07 mbar or less for at least 48 hours while gradually increasing the temperature to room temperature.

A freezing of the bacterial cellulose over several hours before starting the freeze drying (sublimation of water at reduced pressure), allows for obtaining a porous bacterial cellulose having a higher swelling capacity than without a prior freezing step.

According to a further embodiment of the method according to the present invention, cleaning of the body includes contacting the body with an alkaline solution, particularly a sodium hydroxide solution. Furthermore, preferably, cleaning of the body includes rinsing the body with water, particularly multiple times.

Furthermore, in an embodiment of the method, particularly after cleaning of the body, the body is cut to size prior to freezing the body by a laser or by at least one blade.

Further, in an embodiment, after mechanically pressing the body, the body is subjected to a final cut, particularly by a laser or at least one blade.

Particularly, according to an embodiment, the body is formed (e.g. with help of cutting the body, see e.g. above) into a patch, a strip, or into a ring.

A further aspect of the present invention relates to a swellable body produced with the method according to the present invention.

According to a further aspect, the present invention relates to a swellable body consisting of bacterial cellulose, the body including a swelling factor of at least 1000%, particularly at least 1500%, particularly at least 1600%, particularly at least 1700%, particularly at least 1800%, particularly at least 1900%, particularly at least 2000%, particularly at least 2100%, particularly at least 2200%. The swelling factor (in %) is defined by the ration between the thickness after swelling to the thickness before swelling (×100).

According to yet another aspect of the present invention, the invention relates to a medical implant including a swellable body according to one of the aspects of the present invention.

According to an embodiment of the medical implant the swellable body forms a seal of the medical implant (e.g. against paravalvular leakages in a TAVI prosthesis).

According to an embodiment of the medical implant, the swellable body forms at least a portion of a member configured to occlude a cavity (of a patient's anatomy/tissue) such as an atrial auricle.

According to a further embodiment of the medical implant, the medical implant includes a scaffold (such as a stent), wherein the body is fixed to the scaffold (particularly to an outside of the scaffold/stent). Particularly, the body can form an outer skirt of the scaffold or stent, or a portion of such an outer skirt.

The novel material produced with help of the present invention is advantageous in that it allows new technical solutions for the following classes of implants: (1) implants with integrated sealing of potential leaks by attached, highly swellable material layers; (2) implants for filling cavities, consisting of a support structure and a highly swellable, space-demanding material; (3) implants for closing holes or puncture sites, consisting of a plastically deformable support structure and a highly swellable, space-demanding material.

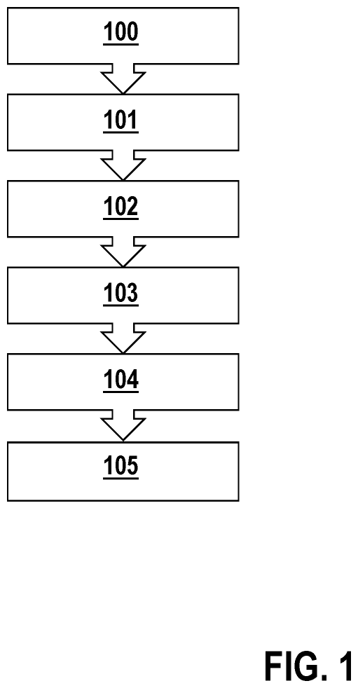

The present invention relates to a method for producing a swellable body 10 including bacterial cellulose, wherein the method includes the steps of (cf. FIG. 1): providing a body 10 made of bacterial cellulose 100, cleaning the body 10 using at least one liquid medium 101, and freeze-drying the body 103.

Particularly, in step 100, according to an embodiment, bacteria from the class Acetobacter xylinum can be used to synthesize the bacterial cellulose, particularly nanocellulose, being used in the method according to the present invention, in a nutrient medium under suitable growth conditions (e.g. standard nutrient medium including acetobacter, 7 days, 28° C., 90% relative humidity). Particularly, the growth of bacterial nanocellulose in the nutrient medium does not take place in the entire volume but always only at the interface with atmospheric oxygen.

According to an example, the bacterial cellulose is generated using a nutrient medium for Acetobacter xylinum having the following composition: 20 g/l glucose, 5 g/l peptone, 5 g/l yeast extract, 2.7 g/l di sodium hydrogen phosphate, 1.5 g citric acid. This culture medium is inoculated with bacteria from the class Acetobacter xylinum. In this nutrient medium, bacterial nanocellulose is formed at typically 26° C. to 30° C. in an incubator over a period of 6 days to 8 days. After 7 days of culture in a dish, a native starting material of about 7 mm to 8 mm thickness is obtained under the above conditions.

The native bacterial cellulose, particularly nanocellulose, can contain large amounts of culture medium as well as the remains of Gram-negative bacteria, which are endotoxins. For this reason, the material is preferably purified in step 101 for typically three days at 80° C. in e.g. 0.1 molar sodium hydroxide solution and a large number of rinsing steps in water. The obtained native patch material 10 can be cut into any shape in step 102 before further processing using a scalpel or a CO₂ laser.

The standard “bacterial nanocellulose” material processed in this way has a very high water content being greater than 98%. Due to the absence of cellulases in the human organism, it is not enzymatically or otherwise degradable as an implant material. In this form, it is already approved for clinical applications.

The removal of water by freezing and freeze drying preserves the structure of the fiber network of the cellulose fibrils, i.e. no irreversible structural change occurs as a result of the formation of new intermolecular hydrogen bonds, so-called cornification. The reason for this is that the water contained in the bacterial cellulose is initially frozen before the process of freeze drying, and the removal of the water takes place by sublimation of the solid water. The absence of capillary forces prevents structural changes.

According to an embodiment, freezing in step 103 includes freezing the material/body 10 for six hours at least −20° C., and 48 hours of drying by lyophilization. Particularly, according to an embodiment, a typical freezing process in step 103 involves freezing the native material at least −20° C. in a freezer for a sufficiently long period, typically at least six hours. This is followed by transfer to a freeze-drying facility with further freezing for at least six hours at −45° C. under atmospheric pressure and drying at e.g. 0.07 mbar for at least 48 hours while gradually increasing the temperature to room temperature. A suitable rate for increasing the temperature is by about 1° C./h. The material 10 obtained is spongy, dimensionally stable and absolutely dry.

The material dried in this way can now be significantly reduced in thickness by pressing or rolling in step 104. Hot pressing at 50° C., 10 N/mm² for 15 minutes typically reduces the thickness by a factor greater than 30. The body/material 10 obtained has a very homogeneous thickness distribution and is mechanically very stable, but still flexible. This allows it to be attached to support structures 2 (e.g. scaffolds, stents etc.) of virtually any shape.

Regarding the above described examples, the pressing parameters can have an influence on the swelling of the freeze-dried body 10 made from bacterial cellulose in that a higher pressing force reduces the swelling capacity, which advantageously also allows the body/material 10 to be adapted to the specific application.

Table 1 shows the values for the swelling behavior of bacterial nanocellulose dried differently before pressing. These samples were cut out from a patch 10 by CO₂ laser and pressed parallel to the growth direction after drying. It can be clearly seen, that surprisingly only the freeze-drying leads to such a strong swelling factor above 22 (i.e. above 2200%).

TABLE 1
Measured thicknesses and calculated swelling behavior of bacterial
nanocellulose dried differently before pressing (7 days standard
culture as patch).

| | | Air drying at room | |
|---|---|---|---|
| Patch material | Freeze drying | temperature | Oven 100° C. |
| Native | 8.50 ± 0.21 mm | 7.98 ± 0.25 mm | 7.78 ± 0.25 mm |
| Dried | 7.41 ± 0.34 mm | 0.16 ± 0.06 mm | 0.16 ± 0.07 mm |
| Pressed | 0.22 ± 0.02 mm | 0.08 ± 0.01 mm | 0.12 ± 0.02 mm |
| Rehydrated | 4.89 ± 0.26 mm | 0.18 ± 0.01 mm | 0.14 ± 0.01 mm |
| Swelling factor | 22.23 | 2.25 | 1.16 |

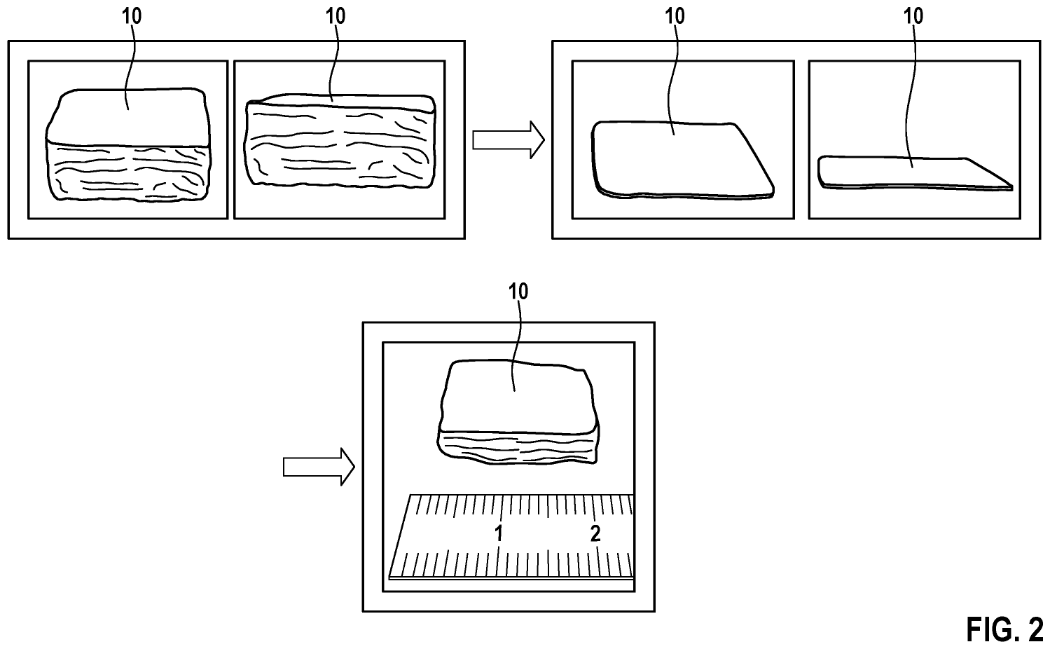

FIG. 2 shows bodies 10 in form of patches of bacterial nanocellulose after drying (48 hours freeze-drying) shown on the left-hand side in the upper row of FIG. 2; after 15 minutes pressing at 10 N/mm² and 50° C. (shown on the right-hand side in the upper row of FIG. 2); and after 24 hours rehydration in water (lower row of FIG. 2). The extreme swelling behavior of the material 10 is also present when pressed perpendicular to the growth direction. For this purpose, rings 10 with 5.0 mm and 2.5 mm wall thickness were cut out from the same native patch material, these were then freeze-dried at the same parameters and then manually rolled thin with a cylindrical rod (5 mm diameter). Table 2 shows the values for the swelling behavior. A swelling factor greater than 1000% is also observed for these specimens.

TABLE 2
Measured thicknesses and calculated swelling factor of rings 10 of
bacterial nanocellulose of different thickness (7 days standard culture
as patch; rings cut out with CO₂ laser).

| Ring material | 5.0 mm wall thickness | | 2.5 mm wall thickness | |
|---|---|---|---|---|
| Native | 5.00 | mm | 2.50 | mm |
| Dried | 3.88 ± 0.01 | mm | 1.48 ± 0.02 | mm |
| Pressed | 0.38 ± 0.01 | mm | 0.18 ± 0.01 | mm |
| Rehydrated | 3.98 ± 0.02 | mm | 1.85 ± 0.02 | mm |

| | | |
|---|---|---|
| Swelling factor | 10.47 | 10.28 |

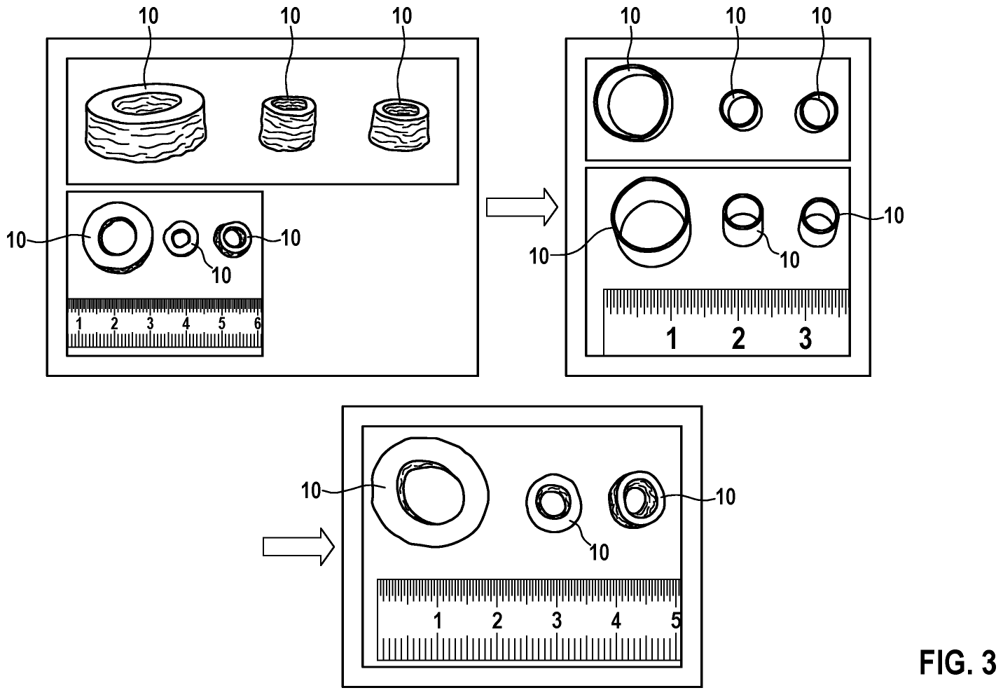

Furthermore, FIG. 3 shows bodies 10 in form of rings of bacterial nanocellulose after 48 hours of freeze drying (left-hand side in the upper row of FIG. 3), after pressing by manual rolling (right-hand side in the upper row of FIG. 3) and after 1 hour of rehydration in water (lower row of FIG. 3).

Swellable bodies 10 according to the invention produced e.g. using the method shown in FIG. 1 can be cut into any shape by final cutting (step 105). For example, rectangular strips can be obtained which, when dry, can be attached to an outside of a stent of a TAVI (transcatheter aortic valve implantation) prosthesis as an outer skirt by conventional suturing. Since the overall system must be dry, this is possible for TAVI prosthesis that use, for example, stabilized dried porcine pericardium as biological material.

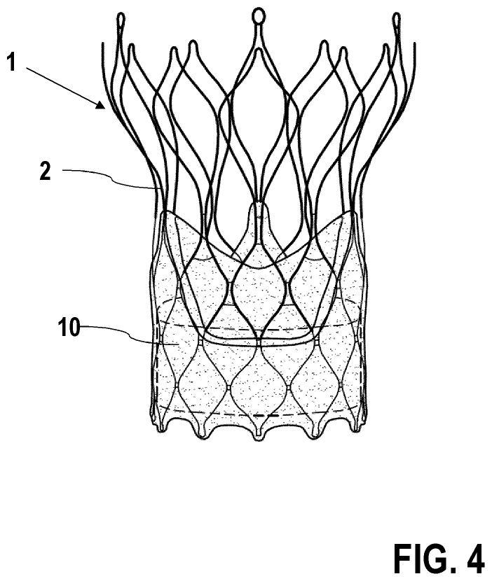

FIG. 4 shows such a TAVI prosthesis 1 including a stent 2 including interconnected struts forming a circumferential cell structure, wherein an outer skirt 10 is fixed to an outside of the stent 2 that corresponds to a patch or annular body formed out of bacterial cellulose being freeze dried according to the present invention.

The application of molded parts made of the material 10 according to the present invention can be directly transferred to other applications. Optionally, the material/swellable body 10 can also be applied to the inner side of the supporting framework 2. Likewise, only parts of the implant surface can be covered with the swellable material/body 10.

Particularly, a swellable body 10 made from bacterial cellulose according to the invention can also be used in an implant for filling cavities. This is due to the fact that the swelling factor of the respective swellable body 10 according to the present invention is extremely high, and a 0.2 mm thick dry body 10 results in a body 10 about 5 mm thick in the swollen state. This can be extended as required by providing a body 10 including several separate layers 10a, 10b of the freeze-dried bacterial cellulose 10. Therefore, the material/body 10 according to the present invention also allows to fill large volumes by minimally invasive, catheter-based implantation of a stent 2 with a swellable body/material 10 appropriately attached to an outside of the stent 2. Stabilization of aneurysms with such a system 1 is conceivable. Advantageously, this principle is also transferable to other applications. Thus, an improvement of implants for the closure of the cardiac ear in the left atrium is possible by using the body/material 10 according to the invention.

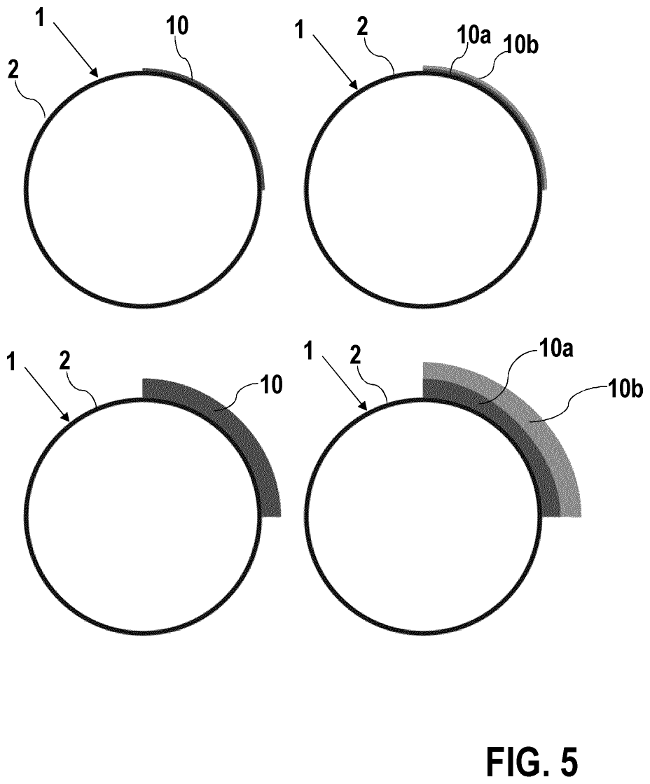

Furthermore, FIG. 5 shows a cross-sectional view of a plastically deformable cylindrical support structure (e.g. scaffold or stent) including a body 10 with one layer (top left) or two layers 10a, 10b (top right) of extremely swellable bacterial cellulose formed according to the method according to the present invention and being locally attached to a sector of the support structure 2 on an outside of the structure 2. By swelling (bottom row), a cavity can be filled in the respective sector due to the extreme increase in thickness of the body 10 attached to the support structure 2. The volume requirement can be expanded as desired by adding two or more layers 10a, 10b of the material (bottom right).

By combining the material/body 10 according to the invention with a deformable retaining structure, a significant improvement of implants for closing holes is conceivable. Examples include closure systems for punctures with a plastically deformable clip structure and closure systems for ventricular septal defects with a plastically super-elastic holding structure made of Nitinol. Due to the extremely strong swelling of the material/body 10, a more secure and stable closure of holes is possible than with non-swellable tissues. The bacterial nanocellulose is also stable over the long term and provides a natural barrier to microorganisms.

In a very general way, the method according to the present invention allows the production of an exceptionally swellable biomaterial that can assume virtually any three-dimensional form in the swollen state. With such a material, enormous technical advantages arise for the realization of implants that (1) incorporate such a body 10 as an integrated seal for leakage or a part thereof, or (2) as an element being capable of filling cavities, or (3) as an element being configured to selectively close openings.

The method according to the present invention can be applied to all types of native bacterial cellulose, i.e., regardless of bacterial strain and cultivation conditions.

Furthermore, advantageously, implementation of the present invention in an approved medical manufacturing process is very straightforward, as established techniques can be used and no other substances have to be introduced into the pure bacterial nanocellulose.

The swelling capacity of the material can be adapted to the requirements of the respective medical implant by the pressing parameters (e.g. pressing pressure, temperature of the body 10 upon pressing, duration of the pressing). Molded parts made from the material 10 produced according to the present invention can be very easily attached to retaining structures 2 using existing methods. Furthermore, bacterial nanocellulose has potential advantages over xenogeneic materials commonly used in biological implants, such as porcine pericardium, in terms of tendency to calcify, homogeneity of material properties, reduced thickness with comparable mechanical properties, production in virtually any shape, stability to biodegradation without additional chemical fixation.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

1. A method for producing a swellable body comprising bacterial cellulose, the method comprising:
providing a body made of bacterial cellulose,
cleaning the body using at least one liquid medium,
freezing the body at atmospheric pressure for at least six hours,
freeze drying the body, and
mechanically pressing the entire body or parts thereof after freeze drying of the body.

2. (canceled)

3. The method according to claim 1, wherein the entire body or parts thereof comprise a temperature of at least 20° C. during the mechanically pressing.

4. The method according to claim 1, wherein the freezing, comprises subjecting the body for at least six hours to a temperature equal to or below −20° C. at atmospheric pressure.

5. The method according to claim 1, wherein the cleaning comprises contacting the body with an alkaline solution.

6. The method according to claim 1, comprising cutting the body to size with a laser or blade prior to the freezing.

7. The method according to claim 1, comprising final cutting the body with a laser or a blade after the mechanically pressing.

8. The method according to claim 1, comprising forming the body into one of: a patch, a strip, and a ring.

9. (canceled)

10. A swellable body consisting of bacterial cellulose, the swellable body comprising a swelling factor of at least 1000%.

11. (canceled)

12. The swellable body according to claim 10 forming, a seal of a medical implant.

13. The swellable bock according to claim 12, forming at least a portion of a member configured to occlude a cavity.

14. The swellable body according to claim 12, wherein the medical implant comprises a scaffold, and the swellable body is fixed to the scaffold.

15. The method according to claim 1, wherein the mechanically pressing comprises applying a pressure of at least 1 N/mm².

16. The method according to claim 1, wherein the mechanically pressing is conducted for at least 15 minutes.

17. The method according to claim 1, wherein the freeze drying comprises subjecting the body to a pressure of 0.07 mbar or less for at least 48 hours while increasing the temperature of the body to room temperature.

18. The method according to claim 5, wherein the alkaline solution comprises a sodium hydroxide solution.

19. The method according to claim 1, wherein the cleaning comprises rinsing the body with water.

20. The swellable body of claim 10, wherein the swelling factor is at least 2000%.

21. The swellable body of claim 20, wherein the swelling factor is at least 2200%.
